(19) United States
(12) Patent Application Publication
(10) Pub. No.: US 2022/0266058 A1
(43) Pub. Date: Aug. 25, 2022

(54) Systems and Methods for Creating Radiation Shields

(71) Applicant: Mayo Foundation for Medical Education and Research, Rochester, MN (US)

(72) Inventors: Justin D. Gagneur, Peoria, AZ (US); Daniel F. Craft, Olathe, KS (US); Michael D. Armstrong, Scottsdale, AZ (US); Jarrod M. Lentz, Phoenix, AZ (US); Madeline G. Foster, Scottsdale, AZ (US)

(21) Appl. No.: 17/184,824

(22) Filed: Feb. 25, 2021

(51) Int. Cl. A61N 5/10; G16H 20/40; G16H 30/40; G16H 50/50; G16H 40/40; B33Y 50/00; B22F 10/22; B33Y 10/00; B22F 10/80; B33Y 80/00

(52) U.S. Cl. CPC A61N 5/10; G16H 20/40; G16H 30/40; G16H 50/50; G16H 40/40; B33Y 50/00; B22F 10/22; B33Y 10/00; B22F 10/80; B33Y 80/00; A61N 2005/1094; B22F 2301/10

(57) ABSTRACT

A method for creating or evaluating a radiation shield for a radiation therapy treatment can include receiving, using one or more computing devices, three-dimensional (3D) imaging data, generating, using the one or more computing devices, a 3D volume of a portion of patient from the 3D imaging data, determining, using the one or more computing devices, a region of interest for receiving radiation therapy for the 3D volume of the portion of the patient, generating, using the one or more computing devices, a 3D model of a radiation shield from the 3D volume of the portion of the patient and the region of interest, the 3D model having an inner surface that contours an exterior surface of the 3D volume, and causing, using the one or more computing devices, a 3D printer to construct a radiation shield from the 3D model of the radiation shield.

### CROSS-REFERENCE TO RELATED APPLICATIONS

N/A.

### STATEMENT REGARDING FEDERALLY SPONSORED RESEARCH

N/A.

### BACKGROUND

Radiation shields are used during radiation therapy treatment to protect healthy tissue that is adjacent a tumor site (or other target tissue) from receiving the radiation therapy beam (e.g., by attenuating the beam) while allowing the tumor site to appropriately receive the radiation therapy beam. Radiation shields are currently formed in ways that are time consuming, labor intensive, and uncomfortable for the patient. Additionally, some radiation shields are formed from materials that are toxic. Thus, it would be desirable to have improved systems and methods for creating radiation shields.

### SUMMARY OF THE DISCLOSURE

Some embodiments of the disclosure provide a computer-implemented method for creating or evaluating a radiation shield for a radiation therapy treatment. The method can include receiving, using one or more computing devices, three-dimensional (3D) imaging data, generating, using the one or more computing devices, a 3D volume of a portion of patient from the 3D imaging data, determining, using the one or more computing devices, a region of interest for receiving radiation therapy for the 3D volume of the portion of the patient, generating, using the one or more computing devices, a 3D model of a radiation shield from the 3D volume of the portion of the patient and the region of interest, the 3D model having an inner surface that contours an exterior surface of the 3D volume, and causing, using the one or more computing devices, a 3D printer to construct a radiation shield from the 3D model of the radiation shield.

In some embodiments, the 3D printer constructs the 3D model from a metal filament that is extrudable through the extruder of the 3D printer.

In some embodiments, the metal filament comprises at least one of: copper; tin; or iron.

In some embodiments, the metal filament includes bronze.

In some embodiments, the method can include determining, using the one or more computing devices, a size and a shape of an aperture based on the region of interest for receiving the radiation therapy, and creating, using the one or more computing devices, the aperture through the 3D model of the radiation shield. In some embodiments, when the radiation shield is interfaced with the patient at least a portion of the aperture aligns with a target region that is to receive radiation therapy.

In some embodiments, the radiation shield is configured to cover at least one critical anatomical structure of the patient. The at least one critical anatomical structure can include an eye of the patient.

In some embodiments, a thickness of the radiation shield is in a range between 10 mm and 20 mm.

In some embodiments, the thickness is substantially 15 mm.

In some embodiments, when constructing the radiation shield with the 3D printer, the 3D printer has printer settings that can include a bed temperature of substantially 60° C., a nozzle temperature of substantially 215° C., an infill percentage of substantially 100 percent, a nozzle speed of substantially 50 mm/s, a layer height of substantially 0.30 mm, a nozzle diameter of substantially 0.8 mm, an infill pattern that is rectilinear, or an extrusion multiplier of substantially 1.08.

In some embodiments, a time duration required by the 3D printer to construct the radiation shield is less than 6 hours.

In some embodiments, the method can include determining, using the one or more computing devices, a theoretical mass for the radiation shield based on the 3D model of the radiation shield and a material that is to be used to construct the 3D model of the radiation shield, determining, using the one or more computing devices, an actual mass of the radiation shield, determining, using the one or more computing devices, a difference between the actual mass of the radiation shield and the theoretical mass of the radiation shield, and determining, using the one or more computing devices, that the radiation shield passes a quality test, based on the difference between the masses being below a threshold mass.

In some embodiments, the method can include receiving, using the one or more computing devices, an image of the radiation shield, and identifying, using the one or more computing devices, each hole in the radiation shield that is larger than a size threshold, the size threshold can be 1 mm. The method can include determining, using the one or more computing devices, that the radiation shield passes the quality test, based on a lack of identifying any hole that exceeds the size threshold.

In some embodiments, the image is a mega-voltage x-ray image. In some embodiments, the image is a kilo-voltage x-ray image.

In some embodiments, the method can include comparing, using the one or more computing devices, an actual thickness of the radiation shield and a theoretical thickness of the 3D model of the radiation shield, the actual thickness and the theoretical thickness sharing a common region of the radiation shield, and determining, using the one or more computing devices, that the radiation shield passes the quality test, based on the comparison of the actual thickness to the theoretical thickness.

In some embodiments, the radiation shield includes a mask to be interfaced with at least a portion of the head of the patient. In other embodiments, the radiation shield can be interfaced with other portions of the patient.

Some embodiments of the disclosure can provide a radiation shield for superficial radiation therapy treatment. The radiation shield can include a first surface and a second surface opposite the first surface, the first surface can contour an exterior surface of a portion of a patient adjacent a region of interest that is to receive superficial radiation therapy, the exterior surface of the patient can include skin of the patient, and an aperture directed through the radiation shield that can correspond to the shape and the size of the region of interest that is to receive radiation therapy. The aperture can align with at least a portion of the region of interest when the radiation shield is interfaced with the exterior surface of the patient. The first surface and the second surface can define a thickness of the radiation shield, the thickness of the radiation shield can be larger than 10 mm. The radiation shield can include at least one of copper, tin, or iron.

In some embodiments, the radiation shield is constructed from a three-dimensional (3D) printer.

In some embodiments, the radiation shield can include a coupling to secure the radiation shield to the exterior surface of the patient. The coupling can include an adhesive disposed on the first surface of the radiation shield.

In some embodiments, when the radiation shield is disposed on the exterior surface of the patient and is configured to receive a radiation therapy beam, the radiation therapy beam including an electron beam for superficial radiation therapy.

Some embodiments of the disclosure provide a method for providing superficial radiation treatment to a patient. The method can include generating a three-dimensional (3D) model of a radiation shield, receiving, using a 3D printer, the 3D model of the radiation shield, constructing a radiation shield using the 3D printer, the 3D printer forming the radiation shield from a metal filament, interfacing an interior surface of the radiation shield directly to the an exterior surface of the patient that includes skin, the interior surface of the radiation shield contouring the exterior surface of the patient, and emitting a radiation therapy beam that provides superficial radiation therapy to the patient, the radiation shield attenuating at least a portion of the radiation therapy beam.

The foregoing and other aspects and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

### BRIEF DESCRIPTION OF THE DRAWINGS

The following drawings are provided to help illustrate various features of non-limiting examples of the disclosure, and are not intended to limit the scope of the disclosure or exclude alternative implementations.

FIG. 1 shows a block diagram providing a schematic illustration of a system for creating, evaluating, and using a radiation shield.

FIG. 2 shows a block diagram of a schematic illustration of a 3D printing system.

FIG. 3 shows a flowchart of a process for creating a radiation shield.

FIG. 4 shows a flowchart of a process for evaluating a quality of a radiation shield.

FIG. 5 shows an image of a table for 3D printer settings for a 3D printer.

FIG. 6 shows four separate portions detonated (a), (b), (c), and (d). Portion (a) of FIG. 6 shows an axial view of a designed radiation shield, portion (b) of FIG. 6 shows the placement locations of the optically stimulated luminescence dosimeter (“OSLDs”) on the phantom, portion (c) of FIG. 6 shows a beam eye view of the digitally rendered radiation shield, and portion (d) of FIG. 6 shows the final printed radiation shield on the phantom.

FIG. 7 shows an image of a table listing the characteristics of the patients treated with the 3D printed radiation shields.

FIG. 8 an image of a table listing the relative radiation transmission for various thicknesses of 3D printed bronze.

FIG. 9 shows a graph of the shield surface enhancement effects for a range of shield aperture sizes and 6 and 9 MeV radiation shields.

FIG. 10 shows a graph of the shield output factors for a range of shield aperture sizes and 6 and 9 MeV radiation shields.

FIG. 11 shows a graph of percent dose measurement of 6 MeV electron beams without shielding, with lead shielding (having a 3 cm shield aperture), and with 3D printed bronze shielding (having a 3 cm shield aperture).

FIG. 12 shows a graph of percent dose measurement of 9 MeV electron beams without shielding, with lead shielding (having a 5 cm shield aperture), and with 3D printed bronze shielding (having a 5 cm shield aperture).

FIG. 13 shows a graph of the beam profile of 6 MeV electron beams without shielding, with lead shielding (having a 3 cm shield aperture), and with 3D printed bronze shielding (having a 3 cm shield aperture).

FIG. 14 shows a graph of the beam profile of 9 MeV electron beams without shielding, with lead shielding (having a 5 cm shield aperture), and with 3D printed bronze shielding (having a 5 cm shield aperture).

FIG. 15 shows an image of a table showing the OSLD results from the phantom measurements.

### DETAILED DESCRIPTION OF THE PRESENT DISCLOSURE

As described above, radiation shields are placed on a patient and are used during radiation therapy to protect the healthy tissue of the patient. For example, for superficial cancers (e.g., of the head and neck) the radiation shield can be placed directly on the skin of the patient. With the radiation shield on, the patient receives a radiation therapy beam (e.g., a megavoltage electron beam) and the radiation shield shapes the radiation therapy beam so that the healthy tissue that surrounds the cancer site receives a significantly attenuated radiation dose (e.g., 5 percent of the dose of the radiation therapy beam), while the cancer receives a radiation dose that is not attenuated by the radiation shield (e.g., a radiation dose that is substantially similar to the dose of the radiation therapy beam).

Although conventional radiation shields made of lead are clinically acceptable and are used widely, they are difficult to manufacture, require a substantial amount of labor, and are uncomfortable for patients. For example, these lead radiation shields are commonly produced by first creating a negative impression of a patient's face (or treatment area) with a flexible thermoplastic sheet, while the patient is in the treatment position (e.g., the position that the patient is to be placed during radiation therapy treatment). The patient must maintain the treatment position while the negative mold hardens, which can take up to 15 minutes. In some cases, the treatment positions can be uncomfortable for patients—especially older patients that have difficulty maintaining the position for such an extended period of time. Additionally, some target locations require even worse conditions. For example, cancers on the head or neck often require the patient to breathe through a straw during the entire negative mold formation process (e.g., because the thermoplastic sheet encapsulates their head, and the patient must be able to breathe during the mold formation process).

After the negative mold formation process is complete, the negative mold is filled with plaster to create a positive model of the patient, which can take an additional 24 hours to harden. Lead is then manually hammered and formed on the positive mold to form the lead radiation shield, which takes an additional 1 to 4 hours of time and labor. The resulting lead radiation shield using this process is not perfectly conformal (e.g., due to the accumulation of errors in each step of the process). In general, this process is time consuming, labor intensive, takes up valuable time, and is uncomfortable for patients. Additionally, this process requires extensive handling of lead, a toxic substance that can be absorbed through the skin.

Some embodiments of the disclosure provide advantages to these issues (and others) by providing improved systems and methods for creating radiation shields. For example, rather than creating molds (e.g., positive and negative molds) and forming the radiation shield against one of these molds, a radiation shield can be digitally rendered from imaging data of the patient, and this digitally rendered radiation shield can be printed by a 3D printer. In this way, because the molds of the patient are avoided, the patients are not required to maintain uncomfortable positions. Additionally, by 3D printing the radiation shield, manual labor and operator skill used to form the lead radiation shield around the positive mold are removed. Still further, metal filaments used by the 3D printer to construct the radiation shield are non-toxic, rather than conventionally used lead.

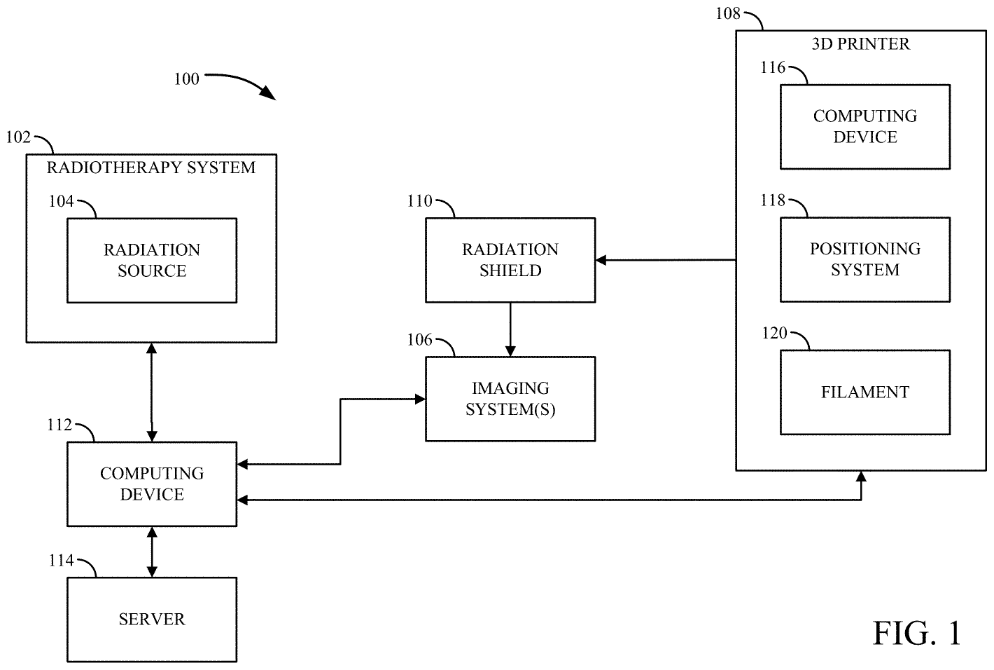

FIG. 1 is a schematic illustration of a system 100 for creating, evaluating, and using a radiation shield. The system 100 can include a radiotherapy system 102 having a radiation source 104, imaging systems 106, a 3D printer 108 that creates a radiation shield 110, a computing device 112 and a server 114. The radiotherapy system 102 can be configured to provide superficial radiation therapy to a patient, and thus can be implemented in different ways. For example, the radiotherapy system 102 (and the radiation source 104) can be configured to provide photon based radiation therapy, or particle based photon therapy. One imaging system of the imaging systems 106 can be configured to acquire imaging data (e.g., 3D imaging data) of the patient of the target region that is to receive radiation therapy. This imaging system can be a computed tomography (“CT”) imaging system, a magnetic resonance imaging system, an optical based imaging system (e.g., a stereoscopic imaging system), etc. A second imaging system of the imaging systems 106 can be configured to acquire imaging data of the radiation shield (e.g., 3D imaging data), which can be used to evaluate a quality of the radiation shield 110 (e.g., if the radiation shield 110 passes a quality test). In some configurations, this imaging system can be an x-ray based imaging system (e.g., a mega voltage x-ray imaging system), an optical based imaging system (e.g., including imaging sensors, cameras, etc.), etc.

As shown, the 3D printer 108 includes a computing device 116, a positioning system 118, and a filament 120. The computing device 116 can be implemented in different ways. For example, the computing device 116 can include typical components used such as a processor, memory, a display, inputs (e.g., a keyboard, a mouse, a graphical user interface, a touch-screen display, etc.), communication devices, etc. In some cases, the computing device 116 can simply be implemented as a processor. The computing device 116 can communicate with other computing devices and systems such as the computing device 112, and can implement some or all of the processes described below. The computing device 116 can implement the functionalities needed to successfully create the radiation shield 110 from a 3D model of the radiation shield 110. For example, the computing device 116 can control the positioning system 118, which includes an extruder, according to various settings of the 3D printer 108 and according to the 3D model of the radiation shield 110. The 3D printer 108 can construct the radiation shield 110 using a filament 120 (e.g., a metal filament). In some configurations, the metal filament can include copper, tin, or iron. In some specific cases, the metal filament is bronze.

The computing device 112 can be implemented in a similar manner as the computing device 116, and in some specific cases can be a laptop computer, a desktop computer, a tablet, a smartphone, a stand-alone computer system, etc. The computing device 112 is in communication with the radiotherapy system 102, the imaging systems 106, the 3D printer 108, and the server 114. Thus, the computing device 112 can control (e.g., transmit instructions to) one or more of these components, can receive data from one or more of these components, and can transmit data to one or more of these components. For example, the computing device 112 can cause the radiotherapy system 102 to turn the radiation source 104 on to emit a superficial radiation beam. As another example, the computing device 112 can create the 3D model of the radiation shield 110 and transmit the 3D model of the radiation shield to the 3D printer 108. As yet another example, the computing device 112 can adjust (or transmit) parameters for the 3D printer 108 to utilize during creation of the radiation shield 110.

In some embodiments, the computing device 112 can, after receiving (e.g., from the server 114) or generating the 3D model of the radiation shield 110, send the 3D model to another computing device in communication with the computing device 112 for post-processing of the 3D model of the radiation shield 110 (e.g., changing the format of the model from a treatment planning computer format to an STL format). In this case, after this other computing device post-processes the 3D model of the radiation shield 110, that computing device can transmit the post-processed 3D model of the radiation shield 110 to the 3D printer 108 for construction of the radiation shield 110.

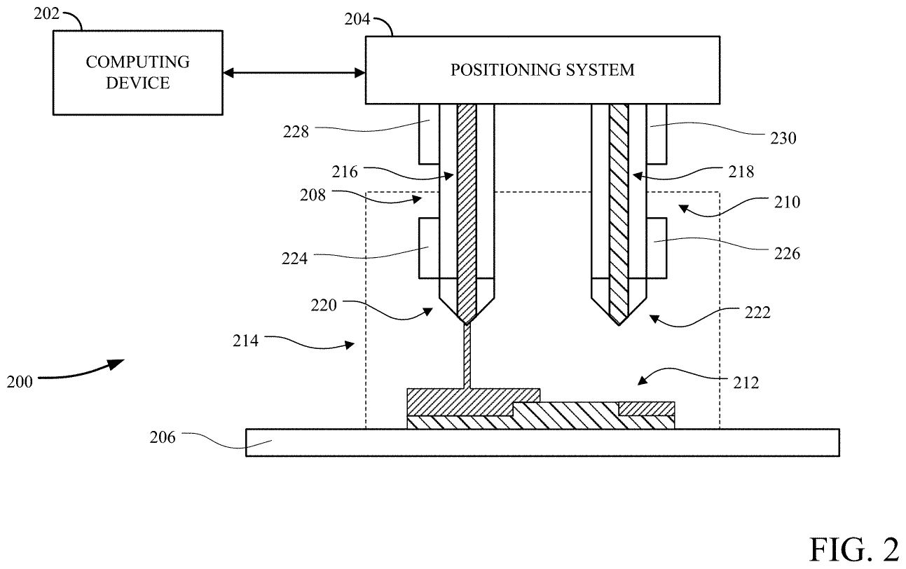

FIG. 2 shows a schematic illustration of a 3D printing system 200, which is a specific implementation of the 3D printer 108. The 3D printing system 200 can include a computing device 202 in communication with a positioning system 204. The positioning system 204 can include a build platform 206, a first extruder 208, and a second extruder 210. The positioning system 204 can move the extruders 208, 210 relative to the build platform 206 in order to fabricate an object 212 while one or both of the extruders 208, 210 deposit build material. In some cases, the build platform 206 can be a rigid and planar surface on which the object 212 is fabricated in a build volume 214. Although shown with two extruders 208, 210, the 3D printing system 200 can alternatively include only a single extruder, or may include more than two extruders. Advantageously, a 3D printing system 200 with two extruders 208, 210 allows for the fabrication of objects, such as the radiation shields described in the present disclosure, using more than one build material type. Alternatively, both extruders 208, 210 could be configured to deposit the same build material type.

Each extruder 208, 210 can include a respective chamber 216, 218 in an interior thereof to receive a build material (e.g., a filament, such as a metal filament), and each extruder 208, 210 can include a respective extrusion tip 220, 222 that extrudes build material. In some cases, each extruder 208, 210 can include a respective heating element 224, 226 to melt the metal filament or other metal build material within the respective chambers 216, 218 for extrusion through the respective extrusion tips 220, 222 in liquid form. In some cases, each extruder 208, 210 can also include a respective motor 228, 230 to force the build material through the respective chambers 216, 218 and through the respective tips 220, 222.

As one example of operation, a build material such as a bronze metal filament can be fed into the chamber 216 from a spool or the like by the motor 228, melted by the heating element 224, and extruded from the extrusion tip 220. By controlling a rate of the motor 228, the temperature of the heating element 224, other process parameters, or combinations thereof, the build material can be extruded, which can impact the overall shape of the object 212.

The positioning system 204 can be generally adapted to three-dimensionally position the extruders 208, 210 and the corresponding extrusion tips 220, 222 such that the extruders 208, 210 deposit build material in accordance with a previously computed object design (e.g., a 3D model of the radiation shield 110). In general, the object may be fabricated by depositing successive layers of material in two-dimensional patterns determined by the computed object design.

The 3D printing system 200 can be operated under the control of the computing device 202 that is in communication, such as wired or wireless communication, with the positioning system 204 including the build platform 206, and other components of the 3D printing system 200. In general, the computing device 202 is operable to control the components of the 3D printing system 200, such as the build platform 206, extruders 208, 210, and positioning system 204 to fabricate the object 212 from the suitable build materials. The computing device 202 can include any combination of software, processing circuitry, or both suitable for controlling the various components of the 3D printing system 200. As an example, the controller may include a microprocessor, microcontroller, application-specific integrated circuit (“ASIC”), programmable gate arrays, and any other digital or analog components. In some embodiments, the computing device 202 can be a processor associated with a personal computer or other computing device that is in communication with the 3D printing system 200. Thus, in some embodiments the computing device 202 can be configured or otherwise programmed to perform the processes described herein.

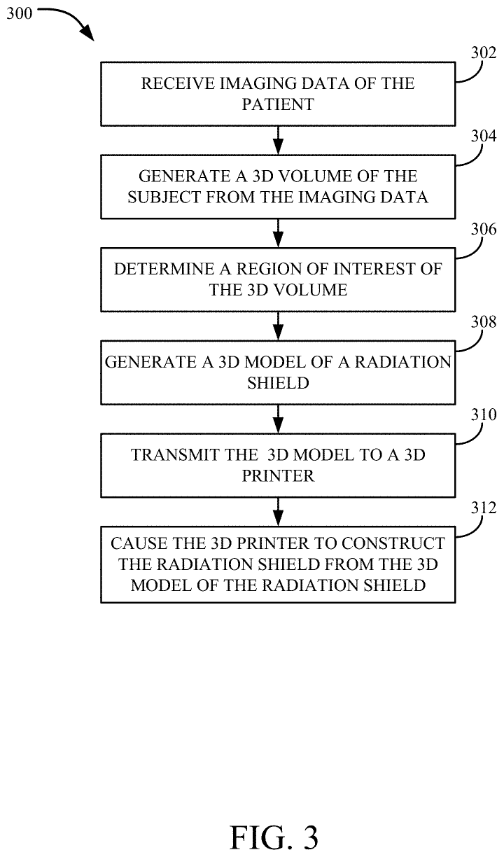

FIG. 3 shows a flowchart of a process 300 for creating a radiation shield, which can be implemented entirely (or partially) using one or more computing devices (e.g., the computing device 112). At 302, the process 300 can include a computing device receiving imaging data of the patient. In some cases, this imaging data can be 3D imaging data (e.g., 3D CT imaging data). The imaging data can be of a portion of the patient that is to receive superficial radiation treatment. For example, the imaging data can be of a head of a patient, an extremity of the patient, etc.

At 304, the process 300 can include a computing device generating a 3D volume of the subject from the imaging data (e.g., received at block 302 of process). The 3D volume includes a region of the subject that is to receive superficial radiation therapy. Thus, this 3D volume can be can various portions of the patient including a head of the patient, an extremity of the patient (e.g., an arm, a leg, etc.), etc. In some embodiments, after the 3D volume of the subject has been generated, the 3D volume can be manipulated, changed, etc. For example, the computing device can receive a user input (e.g., from a user interacting with a computing device) that can truncate the 3D volume of the region of the subject (e.g., remove a portion that is not needed), adjust (e.g., smooth) surfaces of 3D volume, bridge voids or other artifacts in the 3D volume, etc.

At 306, the process 300 can include a computing device determining a region of interest of the 3D volume, which is to receive the superficial radiation therapy. In some cases, the region of interest includes identifiable features, such as peripheral outlines of a superficial tumor and thus the computing device can identify and mark the outline of the region of interest to contour these peripheral outlines of the superficial tumor. In other cases, such as when there are no easily identifiable features, the computing device can receive a user input that identifies the region of interest on the 3D volume. In some cases, this region of interest can be an enclosed surface on the 3D volume, or a perimeter of that enclosed surface.

At 308, the process 300 can include a computing device generating a 3D model of a radiation shield according to the 3D volume of the patient, and the determined region of interest. The 3D model of the radiation shield can span a surface of the 3D volume of the patient that is larger than a surface of the 3D volume that includes the region of interest. In other words, such as when the region of interest is a (superficial) tumor, the surfaces (e.g., interior surface and exterior surface) of the 3D model of the radiation shield are larger than the surface of the tumor. In some cases, the 3D model of the radiation shield can be created by defining the outlines of a surface of the 3D volume of the patient and extruding this surface of the 3D volume of the patient by a thickness. In some embodiments, the entire interior surface of the 3D model of the radiation shield partially (or entirely) contours the exterior surface of the 3D volume. In this way, the exterior surface of the radiation shield, when created, can easily interface with the desired portion of the patient.

In some embodiments, generating the 3D model of the radiation shield includes creating an aperture through the 3D model of the radiation shield. In some cases, the computing device, based on the determined region of interest, can determine a size and a shape of the aperture. In some cases, the peripheral edges of the aperture align exactly with the peripheral edges of the region of interest (e.g., a superficial tumor). In this way, when the radiation shield is constructed and interfaced on the patient, the entire aperture of the radiation shield aligns exactly with a target region that is to receive radiation therapy (e.g., the target region on the patient corresponding to the region of interest on the 3D volume). In some cases, the aperture directed through the radiation shield can be larger than the region of interest (e.g., the region of interest being enclosed by the aperture). In this case, the peripheral edges of the aperture can have a shape that is identical or substantially similar (e.g., deviating by less than 20% of identical) to the region of interest, or the peripheral edges of the aperture can have a shape that is different than the region of interest. For example, the aperture can have a typical geometric shape (e.g., a circle, oval, etc.), while the region of interest can have peripheral edges that define an amorphous shape (e.g., for a tumor). In some configurations, having the aperture (substantially) larger (e.g., deviating by 20%) than the area of the region of interest can ensure that the entire target region (e.g., tumor) is treated by the radiation therapy beam (e.g., ensuring that the interface between the tumor and the healthy tissue is treated with radiation therapy to mitigate resurgence of the tumor).

In some embodiments, the edges that define aperture through the 3D model of the radiation shield can be angled to match (e.g., is substantially identical to) the angle of the intended radiation beam. This can be referred to as substantially matching the divergence of the beam, which can aid in sharpening the penumbra. In this way, sharpening the penumbra can be particularly helpful when the radiation target (e.g., the superficial tumor) is near a critical structure (e.g., the eye of the patient).

In some embodiments, the 3D model of the radiation shield can be generated to span entirely (or partially) over a critical anatomical structure of the patient. For example, the critical anatomical structure is an anatomical structure that is not to receive substantial amounts of radiation therapy, and thus a critical anatomical structure can include, an eye, a reproductive organ, etc. In some configurations, the 3D model of the radiation shield can be generated to span entirely (or partially) over a supporting anatomical structure. For example, the supporting anatomical structure can include a structure that provides stability for the radiation shield mitigating movement between the radiation shield and the patient when the radiation shield is interfaced with the patient. Thus, the supporting anatomical structure can include the nose (e.g., a bridge of the nose), a joint (e.g., the shoulder), etc.

At 310, the process 300 can include a computing device transmitting the 3D model to a 3D printer. In some cases, this can include transmitting the entire computed aided design (“CAD”) file, transmitting build instructions generated from or otherwise based on the 3D model, or transmitting other simplified models. In some cases, a computing device can transmit, along with the 3D model for example, 3D printing parameters that are to be utilized by the 3D printer when constructing the radiation shield from the 3D model of the radiation shield. For example, these 3D printing parameters can include a bed temperature (or in other words the build platform), a nozzle temperature, an infill percentage, a nozzle speed, a layer height (e.g., of a single 2D deposited pattern), a nozzle diameter, an infill pattern, an extrusion multiplier (e.g., the flow rate of extrudable material through the extruder), and a type of extrusion material that is to be used (e.g., a metal filament).

At 312, the process 300 can include a computing device causing the 3D printer to construct the radiation shield from the 3D model of the radiation shield. In some cases, the 3D printer can utilize the received 3D printing parameters and use them while printing the radiation shield, or ensure that they pass the desired parameters. In some cases, the 3D printer can construct the radiation shield using the following parameters a bed temperature of substantially 60° C., a nozzle temperature of substantially 215° C., an infill percentage of substantially 100 percent, a nozzle speed of substantially 50 mm/s, a layer height of substantially 0.30 mm, a nozzle diameter of substantially 0.8 mm, an infill pattern that is rectilinear, and an extrusion multiplier of substantially 1.08.

In some configurations, the thickness of the radiation shield can be in a range between 10 mm and 20 mm, or in particular, the thickness can be substantially (or exactly) 15 mm. In this way, the thickness of the radiation shield is sufficient to attenuate a substantial portion of the radiation therapy beam so that tissues located behind the radiation shield receive an attenuated radiation therapy beam that has 95% less energy than the emitted radiation therapy beam. In some embodiments, the computing device can cause the 3D printer to print the radiation shield in less than 6 hours (or in less than 5 hours). In some cases, the 3D printer settings or the 3D model of the radiation shield (e.g., the thickness of the radiation shield) can be adjusted so that the radiation shield can be printed in less than 6 hours.

In some embodiments, after the radiation shield has been constructed, an adhesive layer can be applied to the interior surface of the radiation shield (e.g., the surface that contacts an exteriors surface of the patient). Additionally, a backing can be placed on the adhesive layer (e.g., a plastic backing) to ensure that the adhesive layer is protected (e.g., while transporting the radiation shield). The adhesive layer can help secure the radiation shield to the patient and ensures the radiation shield does not move during the radiation treatment.

In some embodiments, after the radiation shield has been printed a bolus can be manufactured (e.g., cut) to match the aperture of the radiation shield, or to extend across the aperture of the radiation shield. In some cases, the bolus can be coupled to an interior surface of the radiation shield, and can have a thickness (e.g., substantially 5 mm). In some cases, these boluses, when used with a radiation shield may better protect adjacent tissues and sharpen the radiation field edge on the patient's surface.

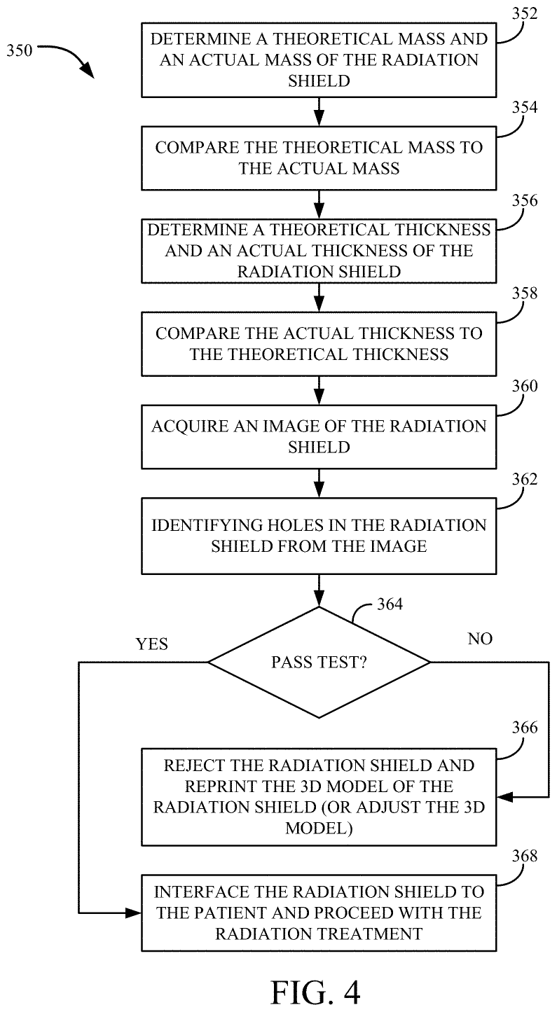

FIG. 4 shows a flowchart of a process 350 for evaluating a quality of a radiation shield, which can be implemented entirely (or partially) using one or more computing devices (e.g., the computing device 112). At 352, the process 350 can include a computing device determining a theoretical mass and an actual mass of the radiation shield. For example, the theoretical mass of the radiation shield can be determined by the volume of the 3D model of the radiation shield, and the type of material used by the 3D printer to construct the radiation shield (e.g., the density of the material). The actual mass, however, can be determined by the constructed radiation shield being placed on a scale or generally weighed. In some cases, a computing device can receive a user input that indicates this actual mass of the radiation shield. In other cases, the scale is in communication with the computing device and the computing device can receive the actual mass directly.

At 352, the process 350 can include a computing device comparing the theoretical mass to the actual mass. In some cases, this can include a computing device calculating the difference between the theoretical mass and the actual mass.

At 356, the process 350 can include a computing device determining a theoretical thickness and an actual thickness of the radiation shield. In some cases, the theoretical thickness is uniform for the entire radiation shield, and thus this value can be used as the theoretical thickness. In other cases, the thickness of the radiation shield can vary from region to region, and thus one thickness from one location (or additional thicknesses at different locations) can be taken. The actual thickness can be determined from measuring a thickness (e.g., with a digital calipers), or other instrument. Similarly to the mass, a computing device can receive a user input that indicates this actual thickness (or thicknesses) of the radiation shield. In other cases, the digital calipers (or other instrument) is in communication with the computing device and the computing device can receive the actual thickness (or thicknesses) directly. Each actual thickness corresponds to the same location of the radiation shield as the theoretical thickness.

At 358, the process 350 can include a computing device comparing each theoretical thickness to each actual thickness. In some cases, this can include a computing device calculating the difference between each theoretical thickness and each actual thickness.

At 360, the process 350 can include a computing device acquiring an image of the radiation shield. In some cases, this can include acquiring multiple images each of different locations of the radiation shield. In some cases, the image can be an x-ray image (e.g., an x-ray megavoltage image). In some embodiments, this can include a computing device acquiring 3D imaging data of the radiation shield.

At 362, process 350 can include a computing device identifying holes in the radiation shield using the image (or images, or 3D volume generated from the 3D imaging data of the radiation shield). In some cases, the computing device can locate each hole within the image, which can be identified by thresholding the image to only include regions within the image having low pixel values (e.g., zero corresponding to black as this indicates in an x-ray image that the radiation beam at this location has been attenuated little to none). In some cases, once the holes have been identified, a computing device can determine which (if any) of the holes are above a particular threshold and store these results, annotate the image with the flagged locations, etc.

At 364, the process 350 can include a computing device determining whether or not the radiation shield passes a quality test. If at 364, the process 350 includes a computing device determining that the quality test has been passed, the process 350 can proceed to block 368 in which the radiation shield is to be interfaced with the patient and the radiation treatment can proceed. If at 364, the process 350 includes a computing device determining that the quality test has not been passed, the process 350 can proceed to block 366 in which the radiation shield is rejected for use in treatment for the patient. In this case, for example, once a computing device determines that the radiation shield has failed the quality test, the computing device can cause the 3D printer to reprint the current 3D model of the radiation shield, or a modified version of the 3D model of the radiation shield (e.g., after a user has modified the previous version of the 3D model of the radiation shield).

In some embodiments, at 364 the test can be failed by failing one of the three criteria, two of the three criteria, or all three criteria. The first criteria can include a computing device comparing the actual mass of the radiation shield to a threshold value (e.g., ±5% of the theoretical mass) and if the actual mass exceeds (e.g., is less than) the threshold value, the computing device can deem the first criteria to have failed. If the actual mass does not exceed the threshold value, the first criteria can be deemed to have passed. In some cases, the difference between the actual mass and the theoretical mass can be divided by the actual mass and compared to a threshold value (e.g., 5%). If the result exceeds the threshold value, the first criteria is deemed to have failed, and if the result does not exceed the threshold the first criteria is deemed to have passed. The second criteria can include a computing device comparing an actual thickness of the radiation shield to a threshold value (e.g., ±5% of the theoretical thickness corresponding to the actual thickness) and if the actual thickness exceeds (e.g., is less than) the threshold value, the computing device can deem the second criteria to have failed. If the actual thickness does not exceed the threshold value, the second criteria can be deemed to have passed. In some cases, the difference between the actual thickness and the theoretical thickness (corresponding to the location of the actual thickness) can be divided by the actual thickness and compared to a threshold value (e.g., 5%). If the result exceeds the threshold value, the second criteria is deemed to have failed, and if the result is does not exceed the threshold value the second criteria is deemed to have passed. In some configurations, where there are multiple pairs that each include an actual thickness and a theoretical thickness, the process above can be implemented to determine the result of each pair. In some cases, if one pair of multiple pairs fails then the second criteria has been deemed failed, and in other cases, only if all pairs fail then the second criteria has been deemed failed

In some embodiments, the third criteria can include a computing device comparing each identified hole of each image to a threshold size (e.g., substantially or identically 1 mm). In some cases, if only one identified hole has exceeded (e.g., is greater than) the threshold size, the third criteria is deemed to have been failed. In other cases, the third criteria can be deemed to have been failed only if the number of identified holes having exceeded the threshold size is greater than a particular number (e.g., 10). If the computing device determines that no identified hole exists that is below a threshold size the third criteria can be deemed to have passed. In other cases, the third criteria can be deemed to have failed if the computing device determines that the number of identified holes having exceeded the threshold size is lower than a particular number. In some embodiments, this multi-criteria approach can catch more problems, ensuring that the radiation shield is of a sufficient quality to be used during radiation treatment.

### EXAMPLES

The following examples have been presented in order to further illustrate aspects of the disclosure, and are not meant to limit the scope of the disclosure in any way. The examples below are intended to be examples of the present disclosure and these (and other aspects of the disclosure) are not to be bounded by theory.

The process of creating radiation shields can be improved and simplified by using 3-dimensional (3D) printing technology instead of manual fabrication techniques. For example, aspects of this disclosure evaluated whether lead equivalent shields could be directly 3D printed using ahigh-density bronze-based filament by assessing the shielding characteristics of 3D-printed bronze (“3DPB”) and determining the clinical viability of 3DPB shields in comparison to conventionally fabricated lead shields.

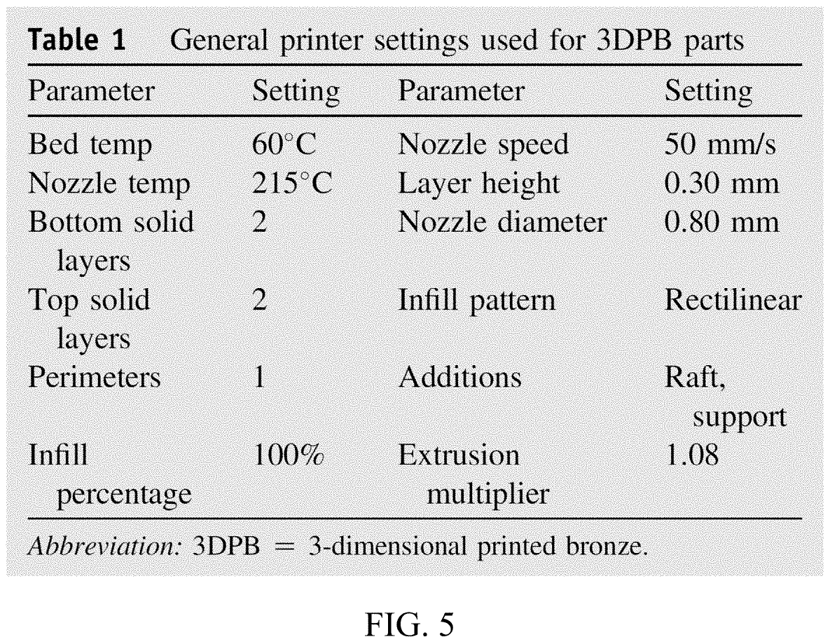

All measurements were performed on a Varian 2100 linear accelerator (Varian Medical Systems, Inc, Palo Alto, Calif.). Film measurements were performed using Gafchromic EBT3 film and analyzed using FilmQA Pro (Ashland, Bridgewater, N.J.). Ion chamber measurements were performed using an Exradin A16 microchamber (Standard Imaging, Inc, Middleton, Wis.), water tank data were recorded with a microDiamond detector (PTW-Freiburg, Freiburg, Germany), and optically stimulated luminescence dosimeter (OSLD) measurements were performed using nanoDots and analyzed using a micro-Starii reader (LANDAUER, Glenwood, Ill.). 3DPB shields were fabricated from colorFabb bronzefill filament (colorFabb, Belfeld, The Netherlands) using an Air-wolf 3D 3D printer (Airwolf 3D, Las Vegas, Nev.). Bronze parts were printed with the settings shown in FIG. 5.

The first task was to determine the necessary thickness of 3DPB to adequately attenuate electron beams by approximately 95%. Three blocks were printed with thicknesses of 5, 10, and 15 mm. Following longstanding practice for measuring attenuation in lead, the attenuation of each 3DPB block was determined by irradiating films [100 cm source to surface distance (SSD), 10 10 cm² cutout] at a 4-mm depth in solid water with and without a 3DPB block placed on the surface for both 6 and 9 MeV electron beams. Film was used so that any areas of nonhomogeneous attenuation due to printing artifacts could be identified. Changes to the dose to the films with and without bronze shielding were determined along the central axis of the beam, under the edges of the shields, and outside the edges of the shields.

Seven flat shields with circular cutouts ranging from 2 to 7 cm in diameter were designed and printed—4 as MeV shields and 3 as 9 MeV shields—to determine the differences between lead and 3DPB shields. Equivalent lead shields were also fabricated. Measurements with shields placed on solid water and an ion chamber placed at D_{ref} were used to measure output factors for lead and bronze shields relative to an unshielded field. Measurements were performed at 100 cm SSD with a 10×10 cm² applicator and an open cutout. A water tank and micro-diamond detector were then used to scan percent depth dose (PDD) curves and beam profiles at a range of depths for 3DPB shields, lead shields, and unshielded fields. Water tank scanning was also done at 100 cm SSD with a 10×10 cm² applicator. The circular cutouts used for the water tank scanning had diameters 2 cm larger than shield apertures.

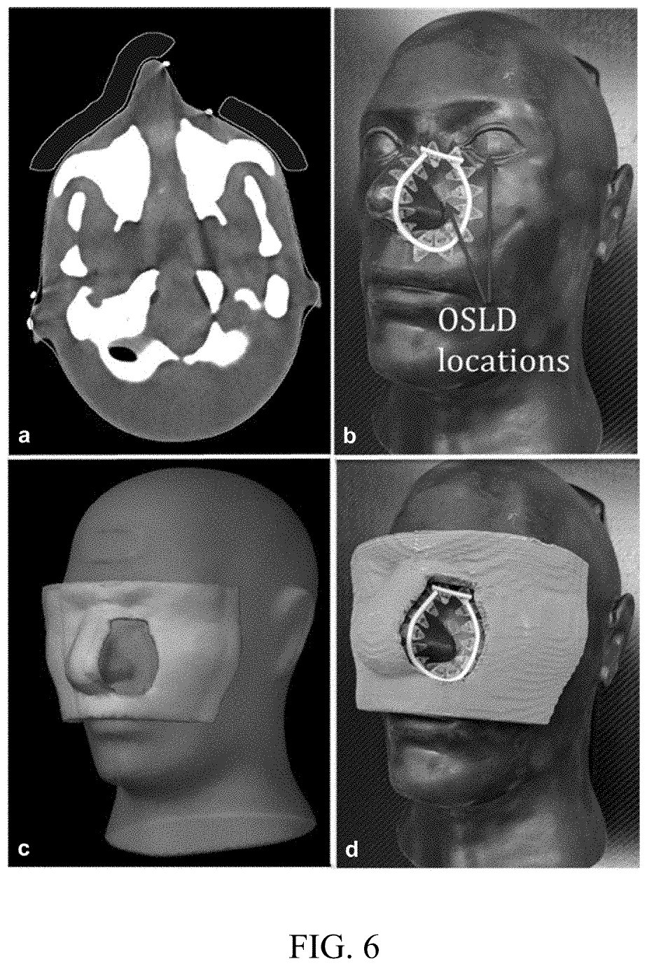

An 11-mm thick (for 6 MeV electrons) and a 16-mm thick (for 9 MeV electrons) shield, whose thicknesses were chosen based on the original attenuation measurements, were designed in the treatment planning system (TPS) for an anthropomorphic head phantom (The Phantom Laboratory, Salem, N.Y.). The shields were designed such that the aperture walls matched the beam angle of the treatment field. The shield structures were exported from the TPS to the 3D Slicer, which was used to transform them into 3D printable .stl files, which were then printed with the previously described settings (see FIG. 5). FIG. 6 shows the design and completed print of the 11-mm thick 6 MeV shield.

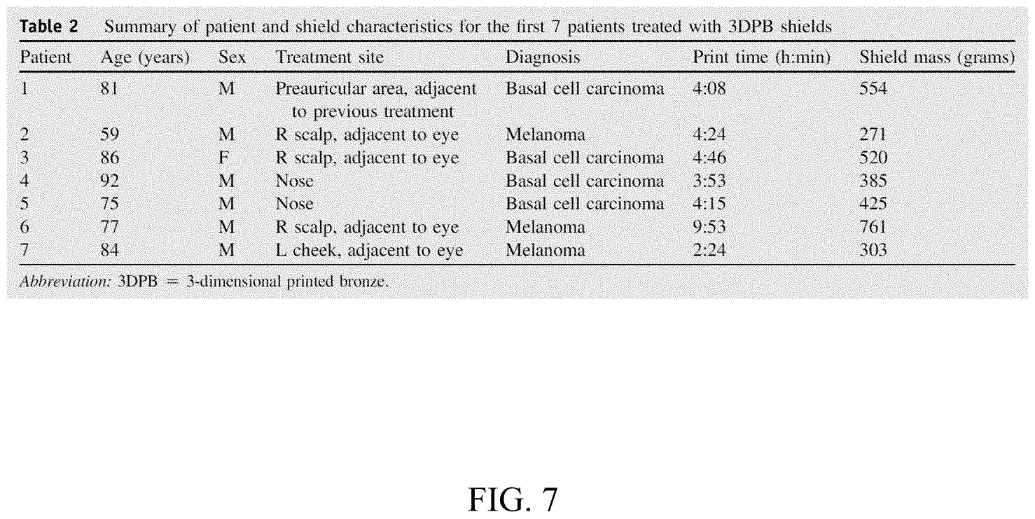

Clinical plans for both 6 and 9 MeV were delivered to the phantom with and without shields present. OSLDs were placed at the 2 indicated positions in portion (b) of FIG. 6 to determine the surface dose enhancement and peripheral attenuation of the shields. Two irradiations were performed of each configuration. 3DPB shields were designed and clinically used for 7 patients. It is reported that this was the first ever clinically used 3DPB shield for an 81-year-old male patient with basal cell carcinoma in his left preauricular area. The shield was designed to partially wrap around this patient's face up to the bridge of his nose so that it would be stable during treatment. It would have been nearly impossible to design a lead shield for this site using our conventional technique. The shield was 11 mm thick, and he was treated to 40 Gy in 10 fractions using 6 MeV electrons prescribed to the 90% isodose line. FIG. 7 shows a summary of patient characteristics for the first 7 patients treated with 3DPB shields.

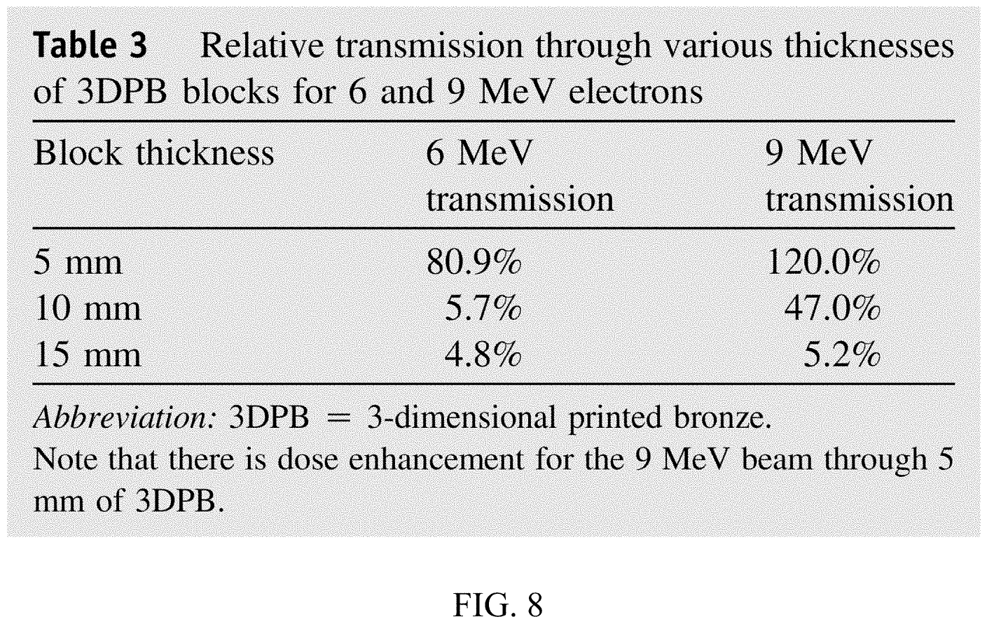

The percent transmission for each block is shown in FIG. 8. It was determined that 10 mm and 15 mm of 3DPB was sufficient shielding for 6 MeV and 9 MeV electrons, respectively. However, subsequent shields and cutouts were designed with an additional millimeter of thickness to account for any printing or design uncertainties. No areas of differential attenuation were noted that could have been caused by printing defects.

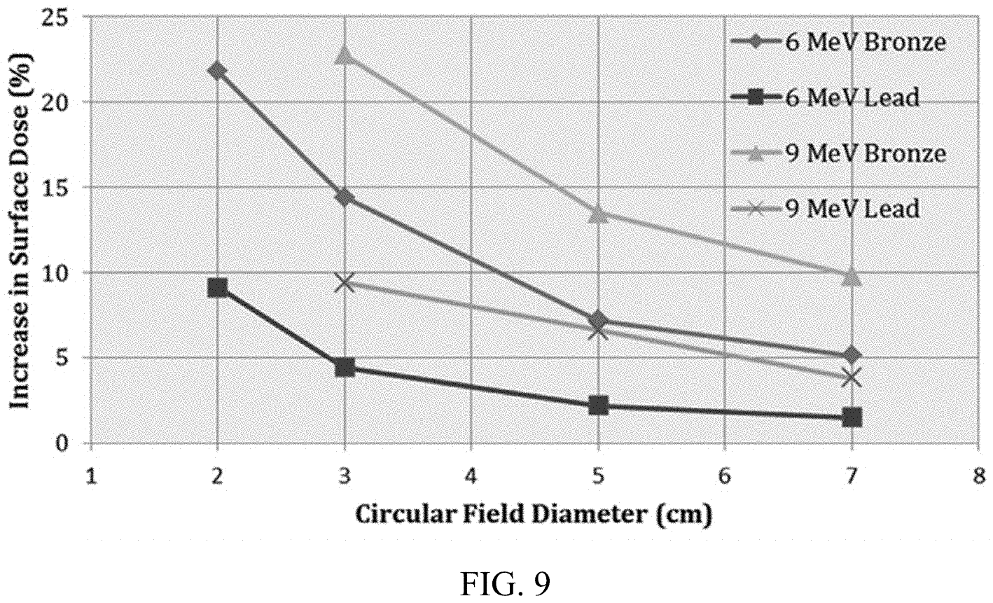

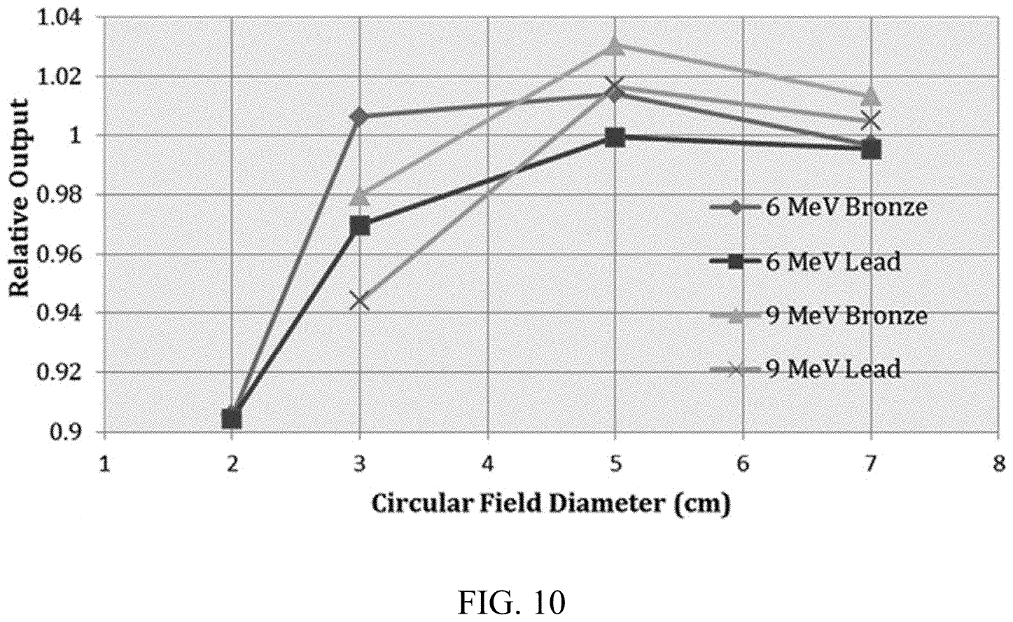

Output factors and surface dose enhancement for lead and 3DPB shields are shown in FIGS. 9 and 10. These results confirm that shields with apertures smaller than the effective range of an electron beam should not be used because beam output starts to be significantly reduced. In practice, this means that 3 cm is the minimum shield aperture size for 6 MeV electrons, and 4.5 cm is the minimum for 9 MeV electrons. Above these shield sizes, all output factors were within 0.97 to 1.03, with an average of 1.006 and 1.020 for 6 MeV 3DPB and lead shields, respectively. For 9 MeV, average output factors were 1.008 and 1.010 for 3DPB and lead shields, respectively.

Surface dose enhancement relative to an unshielded field was determined along the central beam axis using the following equation:

SDE=PDD(1 mm)_{Sheild, D}×OF _{Sheild, D}−PDD(1 mm) _{Open, D}×OF _{Open, D} (1)

where PDD(1 mm) is the PDD measured at 1 mm of shielded and open fields, and OF is the output factor of shielded and open configurations. Surface dose enhancement decreased with increasing aperture size, with lead shields always having lower surface doses than 3DPB shields. For shield apertures larger than the minimum size as previously described, 3DPB shields caused on average 6.2% and 6.5% higher surface doses than lead shields for 6 MeV beams and 9 MeV, respectively.

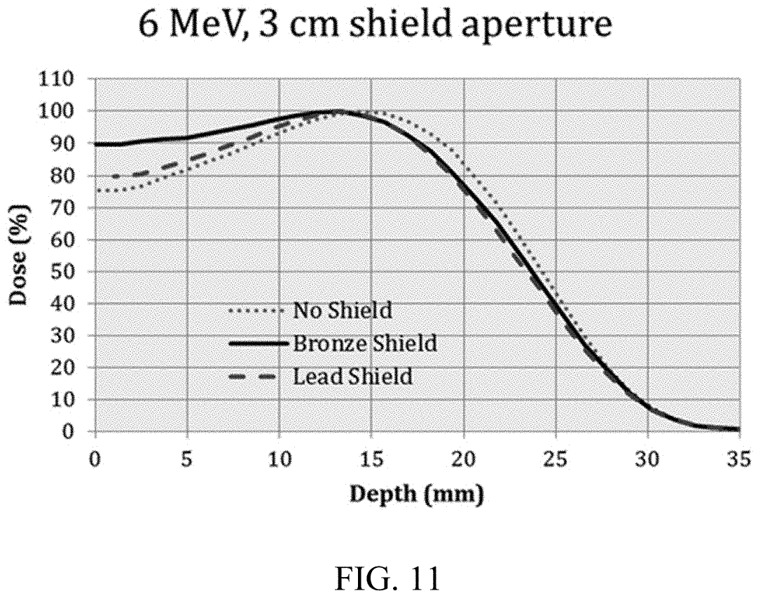

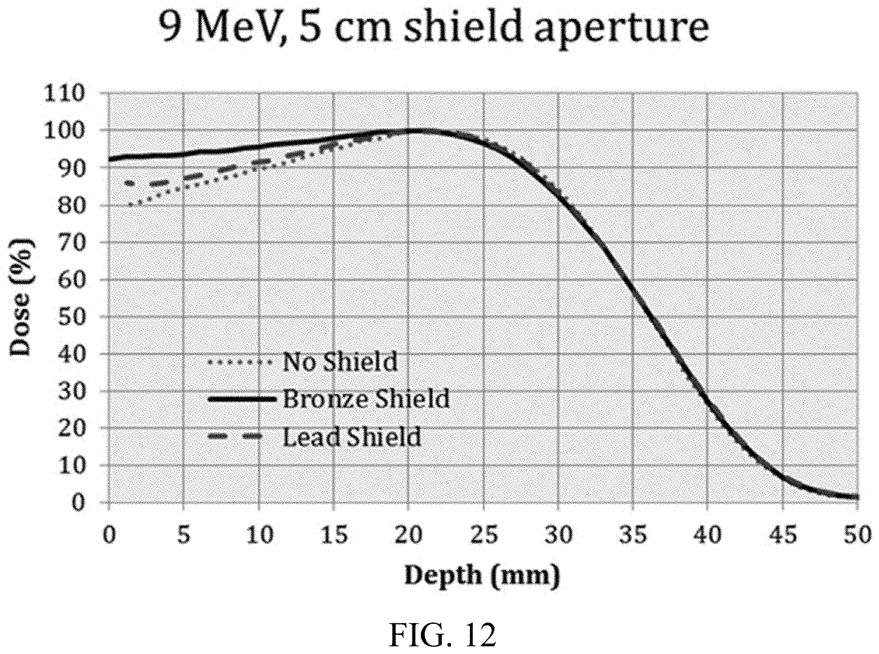

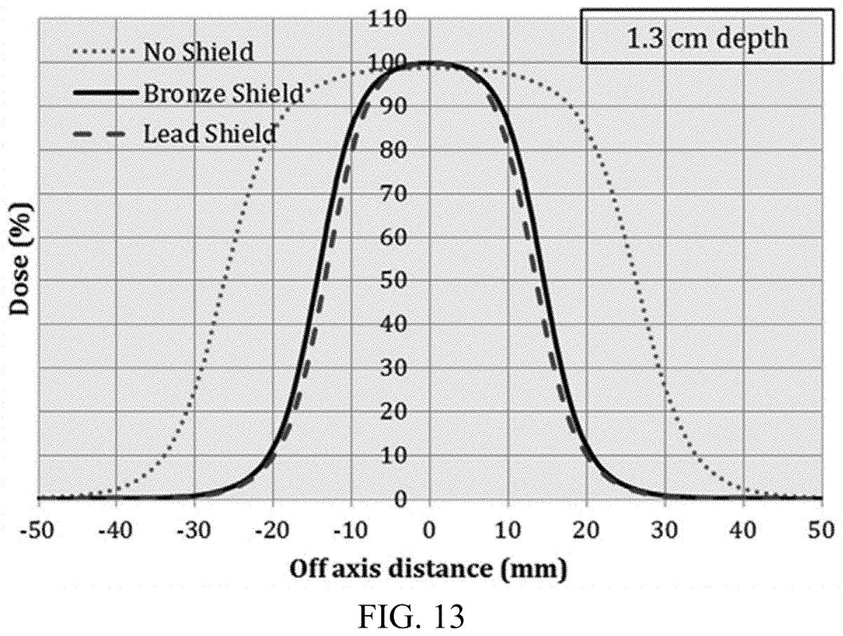

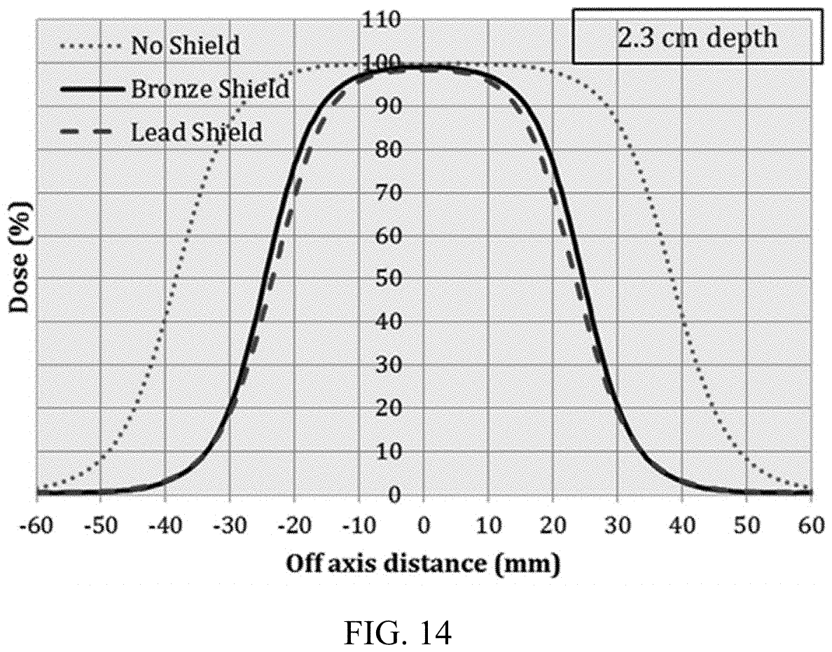

Field profile measurements and PDDs are shown in FIGS. 11-14 for unshielded fields, 3DPB shields, and lead shields. For shield apertures larger than the minimum size, Dₘₐₓ was shifted upstream by an average of 1.2 mm and 1.3 mm for 3DPB and lead shields, respectively, in a 6 MeV beam relative to an unshielded beam. For a 9 MeV beam, Dₘₐₓ was shifted upstream by an average of 0.4 mm and 1.1 mm for 3DPB and lead shields, respectively. Changes to the R₅₀ depth were less than 1.0 mm for all shield and energy configurations. D₉₀, generally the prescription depth, decreased by 1.2 mm and 1.4 mm for 3DPB and lead shields, respectively, in a 6 MeV beam relative to an unshielded beam. For a 9 MeV beam, D₉₀ changed by less than 0.5 mm for both 3DPB and lead shields. The largest differences between PDDs were seen in the increased dose to points shallower than Dₘₐₓ, as described previously. Profiles for lead and 3DPB shields were very similar, with an average difference of 0.7 mm in field width for 6 MeV beams and 1.5 mm for 9 MeV beams. Penumbras were also similar between lead and 3DPB fields (within 1 mm for all shields and energies), and penumbras were always smaller for shielded fields unshielded fields, as expected.

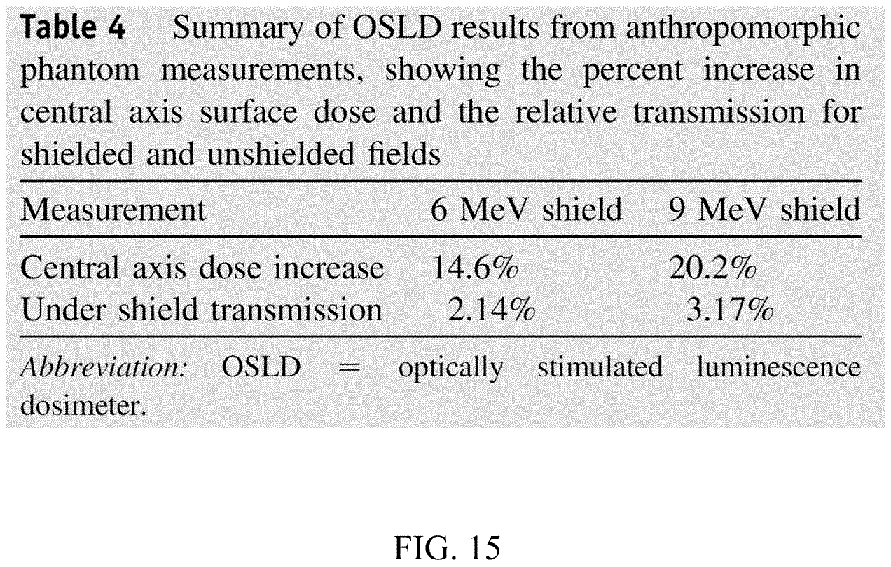

Each shield took approximately 30 minutes to design and less than 6 hours to print. Owing to their high density, the shields could not be CT imaged, but based on visual examination they fit into place on the phantom very well. As expected, the central axis OSLD measurements showed an increase in dose relative to unshielded fields, and the measurement point near the eye showed a dramatically reduced dose relative to unshielded fields. FIG. 15 summarizes these results, which were consistent with the results obtained from water tank scanning data.

All 7 shields were successfully designed and printed according to the established procedures. The average print-time was 4 hours and 49 minutes, and the average shield mass was 460 grams. They fit the patients well based on visual inspection, were easy to place, and were deemed clinically acceptable by the physician. Target coverage and organ at risk protection were confirmed by visual inspection of the shield on the patient compared with the placement in the treatment planning software, and the patients reported no issues with comfort. Cutting and fitting bolus into the apertures was simple and straight-forward.

A new technique for fabricating patient-specific, on-skin electron shields was proposed and evaluated. The previous clinical workflow of producing lead shields was time consuming, labor intensive, and uncomfortable for patients. The newly proposed technique using 3DPB shields is less time consuming, less labor intensive, and does not require patients to undergo an uncomfortable molding process. This is the first study to assess directly 3D printed radiation shields, and the first reported clinical use of 3DPB shields on cancer patients.

Although differences in dosimetry was noted between lead and bronze shields in specific scenarios, they were generally very similar. For shield apertures larger than the approximate range of electrons (e.g., 3 cm for 6 MeV electrons and 4.5 cm for 9 MeV electrons), the change in PDD beyond Dₘₐₓ was small. On-skin shielding has generally been assumed to have no significant effect on the beam output, and measured output factors for 3DPB shields were closer to this assumed value of 1.0 than lead shields. However, both were within a few percent for relevant field sizes. Attenuation of the primary beam under the shields was essentially equivalent for lead and 3DPB. The surface dose, especially for the initial 5 mm, displayed the largest difference between lead and 3DPB shields, but this dosimetric difference was generally not concerning for 2 reasons. First, nearly all patients are treated with at least 5 mm of bolus, so the majority of the surface enhancement does not occur in the patient. Second, the surface of the patient is part of the actual radiation target, and a dose is frequently prescribed to the 90% isodose line to account for differences in the biologic effects of electrons compared with standard kV photons. One may or may not want to compensate for this small increase in surface dose when prescribing electron treatments using 3DPB shields.

Some of the advantages of 3DPB shields over lead shields are not dosimetric related, but are rather workflow related. Printing a custom shield changes the simulation and shield fabrication treatment workflow processes. First, the simulation process is shorter and more comfortable for patients. Previously, custom molding masks were made of patients with sheets of thermoplastic. The process required patients to breathe through straws because the mask covers their face, and it required additional set-up and alignment time. 3DPB shields only require the regular treatment planning CT images (or other images). In total, the new process requires 30 to 45 minutes less in simulation, which opens up the schedule and frees up time for physicians, therapists, and physicists. The treatment planning process is the exact same, so no changes are required. The shield fabrication process is changed. Previously, the mask made during simulation was used to pour a plaster mold, which required 24 hours to harden. A lead shield was then custom-fabricated with 1 to 4 hours of manual fabrication time. The new 3DPB workflow requires 30 to 60 minutes of design and preparation work by a physicist to plan the shield, and then prints in 3 to 8 hours, depending on size, with no additional supervision required. After printing, approximately 15 minutes of quality assurance is required on the shield. The total turnaround time is faster for a 3D printed shield and requires less labor. We estimated that using a 3DPB shield instead of a lead shield saves a substantial amount in direct costs for each treated patient, not including the cost of occupying a CT simulator for additional time. It also improves workflows and is more comfortable for patients. This, however, does not factor in the initial cost of a 3D printer.

Quality assurance (QA) is required for each 3DPB shield and is an important part of ensuring 3D printed materials are used safely. There are 3 potential concerns that QA is designed to evaluate: improper design, global print failure, or local print failures. Improper design of a shield is generally a human error, and would include things like using the wrong thickness, opening the aperture to the wrong target, or failing to account for discrepancies between the patient's external contour and mask positions. These errors are checked for during the plan check by reviewing the shape of the intended shield and by making a simple measurement of the thickness of the final printed shield to make sure it matches. Global print failures are errors in print settings or actual mechanical failure of the printer to extrude the correct amount of bronze throughout the shield. This could result in a shield that externally looks fine, but is a much lower or higher density than intended. This is assessed by noting and then measuring the mass of the part immediately after printing on a calibrated scale. If the density is not within 5% of the intended range, the part fails. Local print failures are errors that may occur if the printer becomes temporarily jammed, allowing small holes or pockets to form inside the shield. These could be very small, so they would not be discovered by the density check. A mega-voltage x-ray (“MV”) image is taken of each part to assess for potential interior defects, and any unexplained voids would cause the device to fail. The ability of our system to catch errors like these was assessed by intentionally creating several holes of various sizes in shields and then taking MV images. Voids can be detected in these images that are much smaller than are clinically relevant, that is, smaller than the 1 mm extra shielding thickness buffer. The entire process of QA, which includes measuring the thickness, measuring the mass, and taking an MV image, can be performed in less than 15 minutes. A standardized report form is used to record the results, including the MV image, which is uploaded to the patient's chart.

In conclusion, the disclosure has shown that 3DPB shields are a clinically viable alternative to lead shields when used for the treatment of superficial lesions with electrons. 3D printed shields reduce the time needed for simulation, decrease patient discomfort, increase conformity to the patient's face, and do not require working with toxic lead. Overall, 3DPB shields appear to be clinically superior to lead shields in cost, comfort, and efficiency.

Custom-fabricated lead shields are often used for superficial radiation treatments to reduce radiation doses to adjacent healthy tissue. However, the process for fabricating these lead shields is time consuming, labor intensive, and uncomfortable for patients. Some embodiments of the present disclosure address these issues by providing patient-specific shields that can be 3-dimensionally (“3D”) printed from a high-density bronze-based filament. This study assessed the shielding characteristics of 3D-printed bronze (“3DPB”) shields, demonstrated their clinical viability, and reported the first ever published case of a patient treated with a 3DPB shield. For example, the transmission of 6 and 9 MeV electron beams were measured through varying thicknesses of 3DPB. Additionally, the percent depth doses and beam profiles were measured for both flat 3DPB shields and equivalent lead shields to determine surface dose enhancement, output factors, and field widths. After, two 3DPB shields were designed and fabricated for an anthropomorphic phantom, and phantom measurements were performed using optically stimulated luminescence dosimeters and film. Finally, 3DPB shields were used during the treatment of skin lesions for 7 different patients. As shown, ten and 15 mm of 3DPB were sufficient to shield 6 and 9 MeV electrons, respectively, by 95%. The 3DPB and lead shields also had nearly identical beam widths (within 1%). Output factors were on average within 0.8% for bronze shields and 1.2% for lead shields relative to an unshielded field. The skin enhancement for bronze was higher than for lead by an average of 6.3%. Phantom measurements using 3DPB shields generally showed less than 3% transmission of the primary beam under the 3DPB shield. The patients' shields fit as designed and were all deemed clinically acceptable by their physicians. In summary, the 3DPB shields fit better than lead shields, were easier to design and manufacture, and have similar dosimetric properties. 3DPB shields are a viable clinical option for patient-specific superficial shielding.

The present disclosure has described one or more preferred embodiments, and it should be appreciated that many equivalents, alternatives, variations, and modifications, aside from those expressly stated, are possible and within the scope of the invention.

It is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

As used herein, unless otherwise limited or defined, discussion of particular directions is provided by example only, with regard to particular embodiments or relevant illustrations. For example, discussion of “top,” “front,” or “back” features is generally intended as a description only of the orientation of such features relative to a reference frame of a particular example or illustration. Correspondingly, for example, a “top” feature may sometimes be disposed below a “bottom” feature (and so on), in some arrangements or embodiments. Further, references to particular rotational or other movements (e.g., counterclockwise rotation) is generally intended as a description only of movement relative a reference frame of a particular example of illustration.

In some embodiments, aspects of the disclosure, including computerized implementations of methods according to the disclosure, can be implemented as a system, method, apparatus, or article of manufacture using standard programming or engineering techniques to produce software, firmware, hardware, or any combination thereof to control a processor device (e.g., a serial or parallel general purpose or specialized processor chip, a single- or multi-core chip, a microprocessor, a field programmable gate array, any variety of combinations of a control unit, arithmetic logic unit, and processor register, and so on), a computer (e.g., a processor device operatively coupled to a memory), or another electronically operated controller to implement aspects detailed herein. Accordingly, for example, embodiments of the disclosure can be implemented as a set of instructions, tangibly embodied on a non-transitory computer-readable media, such that a processor device can implement the instructions based upon reading the instructions from the computer-readable media. Some embodiments of the disclosure can include (or utilize) a control device such as an automation device, a special purpose or general purpose computer including various computer hardware, software, firmware, and so on, consistent with the discussion below. As specific examples, a control device can include a processor, a microcontroller, a field-programmable gate array, a programmable logic controller, logic gates etc., and other typical components that are known in the art for implementation of appropriate functionality (e.g., memory, communication systems, power sources, user interfaces and other inputs, etc.).

The term “article of manufacture” as used herein is intended to encompass a computer program accessible from any computer-readable device, carrier (e.g., non-transitory signals), or media (e.g., non-transitory media). For example, computer-readable media can include but are not limited to magnetic storage devices (e.g., hard disk, floppy disk, magnetic strips, and so on), optical disks (e.g., compact disk (CD), digital versatile disk (DVD), and so on), smart cards, and flash memory devices (e.g., card, stick, and so on). Additionally it should be appreciated that a carrier wave can be employed to carry computer-readable electronic data such as those used in transmitting and receiving electronic mail or in accessing a network such as the Internet or a local area network (LAN). Those skilled in the art will recognize that many modifications may be made to these configurations without departing from the scope or spirit of the claimed subject matter.

Certain operations of methods according to the disclosure, or of systems executing those methods, may be represented schematically in the FIGS. or otherwise discussed herein. Unless otherwise specified or limited, representation in the FIGS. of particular operations in particular spatial order may not necessarily require those operations to be executed in a particular sequence corresponding to the particular spatial order. Correspondingly, certain operations represented in the FIGS., or otherwise disclosed herein, can be executed in different orders than are expressly illustrated or described, as appropriate for particular embodiments of the disclosure. Further, in some embodiments, certain operations can be executed in parallel, including by dedicated parallel processing devices, or separate computing devices configured to interoperate as part of a large system.

As used herein in the context of computer implementation, unless otherwise specified or limited, the terms “component,” “system,” “module,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, unless otherwise defined or limited, ordinal numbers are used herein for convenience of reference based generally on the order in which particular components are presented for the relevant part of the disclosure. In this regard, for example, designations such as “first,” “second,” etc., generally indicate only the order in which the relevant component is introduced for discussion and generally do not indicate or require a particular spatial arrangement, functional or structural primacy or order.

As used herein, unless otherwise defined or limited, directional terms are used for convenience of reference for discussion of particular figures or examples. For example, references to downward (or other) directions or top (or other) positions may be used to discuss aspects of a particular example or figure, but do not necessarily require similar orientation or geometry in all installations or configurations.

This discussion is presented to enable a person skilled in the art to make and use embodiments of the disclosure. Various modifications to the illustrated examples will be readily apparent to those skilled in the art, and the generic principles herein can be applied to other examples and applications without departing from the principles disclosed herein. Thus, embodiments of the disclosure are not intended to be limited to embodiments shown, but are to be accorded the widest scope consistent with the principles and features disclosed herein and the claims below. The following detailed description is to be read with reference to the figures, in which like elements in different figures have like reference numerals. The figures, which are not necessarily to scale, depict selected examples and are not intended to limit the scope of the disclosure. Skilled artisans will recognize the examples provided herein have many useful alternatives and fall within the scope of the disclosure.

Various features and advantages of the disclosure are set forth in the following claims.

What is claimed is:

1. A computer-implemented method for creating or evaluating a radiation shield for a radiation therapy treatment, the method comprising:
receiving, using one or more computing devices, three-dimensional (3D) imaging data;
generating, using the one or more computing devices, a 3D volume of a portion of patient from the 3D imaging data;
determining, using the one or more computing devices, a region of interest for receiving radiation therapy for the 3D volume of the portion of the patient;
generating, using the one or more computing devices, a 3D model of a radiation shield from the 3D volume of the portion of the patient and the region of interest, the 3D model having an inner surface that contours an exterior surface of the 3D volume; and
causing, using the one or more computing devices, a 3D printer to construct a radiation shield from the 3D model of the radiation shield.

2. The method of claim 1, wherein the 3D printer constructs the 3D model from a metal filament that is extrudable through the extruder of the 3D printer.

3. The method of claim 2, wherein the metal filament comprises at least one of: copper; tin; or iron.

4. The method of claim 3, wherein the metal filament includes bronze.

5. The method of claim 1, further comprising:
determining, using the one or more computing devices, a size and a shape of an aperture based on the region of interest for receiving the radiation therapy; and
creating, using the one or more computing devices, the aperture through the 3D model of the radiation shield, and
wherein when the radiation shield is interfaced with the patient at least a portion of the aperture aligns with a target region that is to receive radiation therapy.

6. The method of claim 1, wherein the radiation shield is configured to cover at least one critical anatomical structure of the patient, and
wherein the at least one critical anatomical structure includes an eye of the patient.

7. The method of claim 1, wherein a thickness of the radiation shield is in a range between 10 mm and 20 mm.

8. The method of claim 7, wherein the thickness is substantially 15 mm.

9. The method of claim 1, wherein when constructing the radiation shield with the 3D printer, the 3D printer has printer settings that include:
a bed temperature of substantially 60° C.;
a nozzle temperature of substantially 215° C.;
an infill percentage of substantially 100 percent;
a nozzle speed of substantially 50 mm/s;
a layer height of substantially 0.30 mm;
a nozzle diameter of substantially 0.8 mm;
an infill pattern that is rectilinear; or
an extrusion multiplier of substantially 1.08.

10. The method of claim 1, wherein a time duration required by the 3D printer to construct the radiation shield is less than 6 hours.

11. The method of claim 1, further comprising:
determining, using the one or more computing devices, a theoretical mass for the radiation shield based on the 3D model of the radiation shield and a material that is to be used to construct the 3D model of the radiation shield;
determining, using the one or more computing devices, an actual mass of the radiation shield;
determining, using the one or more computing devices, a difference between the actual mass of the radiation shield and the theoretical mass of the radiation shield; and
determining, using the one or more computing devices, that the radiation shield passes a quality test, based on the difference between the masses being below a threshold mass.

12. The method of claim 11, further comprising:
receiving, using the one or more computing devices, an image of the radiation shield; and
identifying, using the one or more computing devices, each hole in the radiation shield that is larger than a size threshold, the size threshold being 1 mm; and
determining, using the one or more computing devices, that the radiation shield passes the quality test, based on a lack of identifying any hole that exceeds the size threshold.

13. The method of claim 12, wherein the image is a mega-voltage x-ray image.

14. The method of claim 12, further comprising:
comparing, using the one or more computing devices, an actual thickness of the radiation shield and a theoretical thickness of the 3D model of the radiation shield, the actual thickness and the theoretical thickness sharing a common region of the radiation shield; and
determining, using the one or more computing devices, that the radiation shield passes the quality test, based on the comparison of the actual thickness to the theoretical thickness.

15. The method of claim 1, wherein the radiation shield includes a mask to be interfaced with at least a portion of the patient's head.

16. A radiation shield for superficial radiation therapy treatment, the radiation shield comprising:
a first surface and a second surface opposite the first surface, the first surface contouring an exterior surface of a portion of a patient adjacent a region of interest that is to receive superficial radiation therapy, the exterior surface of the patient including skin of the patient;
an aperture directed through the radiation shield that corresponds to the shape and the size of the region of interest that is to receive radiation therapy, the aperture aligning with at least a portion of the region of interest when the radiation shield is interfaced with the exterior surface of the patient, and
the first surface and the second surface defining a thickness of the radiation shield, the thickness of the radiation shield being larger than 10 mm, and
the radiation shield comprising at least one of: copper, tin, or iron.

17. The radiation shield of claim 16, wherein the radiation shield is constructed from a three-dimensional (3D) printer.

18. The radiation shield of claim 16, further comprising a coupling to secure the radiation shield to the exterior surface of the patient, and
wherein the coupling includes an adhesive disposed on the first surface of the radiation shield.

19. The radiation shield of claim 16, wherein when the radiation shield is disposed on the exterior surface of the patient and is configured to receive a radiation therapy beam, the radiation therapy beam including an electron beam for superficial radiation therapy.

20. A method for providing superficial radiation treatment to a patient, the method comprising:
generating a three-dimensional (3D) model of a radiation shield;
receiving, using a 3D printer, the 3D model of the radiation shield;
constructing a radiation shield using the 3D printer, the 3D printer forming the radiation shield from a metal filament;
interfacing an interior surface of the radiation shield directly to the an exterior surface of the patient that includes skin, the interior surface of the radiation shield contouring the exterior surface of the patient; and
emitting a radiation therapy beam that provides superficial radiation therapy to the patient, the radiation shield attenuating at least a portion of the radiation therapy beam.
